(19) United States
(12) Patent Application Publication
(10) Pub. No.: US 2023/0242493 A1
(43) Pub. Date: Aug. 3, 2023

(54) PROCESSES FOR MAKING TETRAZOLINONE COMPOUNDS

(71) Applicant: BASF SE, Ludwigshafen (DE)

(72) Inventors: Birgit Gockel, Ludwigshafen (DE); Volker Maywald, Ludwigshafen (DE); Manfred Ehresmann, Ludwigshafen (DE); Daniel Saelinger, Ludwigshafen (DE); Roland Goetz, Ludwigshafen (DE); Jan Haller, Leverkusen (DE)

(21) Appl. No.: 17/766,286

(22) PCT Filed: Oct. 20, 2020
(86) PCT No.: PCT/EP2020/079416

§ 371 Date: Apr. 4, 2022

(30) Foreign Application Priority Data
Oct. 24, 2019 (EP) 19205137.3

(51) Int. Cl. C07D 257/04

(52) U.S. Cl. CPC C07D 257/04

(57) ABSTRACT

Process for making a compound represented by formula (3), comprising the following steps: a) reacting an azide compound with a compound represented by formula (1) to yield compound (2), compound (2) being a compound represented by formula (2) or its salts, b) methylating compound (2) obtained in step a) using a methylating agent to obtain compound (3), wherein R¹ denotes a hydrocarbon rest, and wherein in step a) a solvent system A and in step b) a solvent system B is used, wherein both solvent systems A and B comprise one dipolar aprotic solvent or a mixture of dipolar aprotic solvents as the main component, and wherein steps a) and b) are carried without isolating the compound (2) obtained in step a).

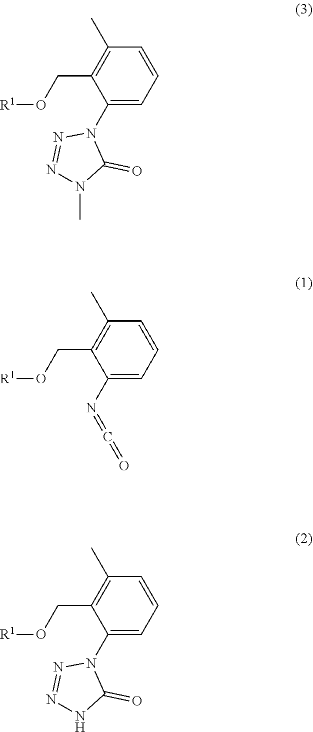

The present invention is directed to processes for making a compound represented by formula (3)

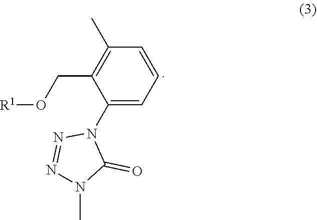

Compounds (3) are important intermediates, for example for the synthesis of pesticides, as it is known for example from WO 2013/162072.

There is a continued need for processes for their manufacture that are easy and economical to carry out and produce compound (3) in high yields and high purity.

JP 2016-113426 discloses a two-step process for producing 1-[2-(methoxymethyl)-3-methyl-phenyl]-4-methyl-tetrazol-5-one by reacting first an isocyanate compound (1) with sodium azide in DMF in the presence of aluminum trichloride to 4-[2-(methoxymethyl)-3-methyl-phenyl]-1H-tetrazol-5-one (2), which—in a second step—is methylated in acetone in the presence of a base to 1-[2-(methoxymethyl)-3-methyl-phenyl]-4-methyl-tetrazol-5-one (3).

It was the objective of the present invention to provide an economic process for the preparation of compound (3) in high yields and high purity.

Herein, compounds represented by formula (1) are denoted as “compound (1)”. Compounds represented by other formulae are denoted accordingly.

It has now been found that this objective can be achieved by processes for making a compound represented by formula (3)

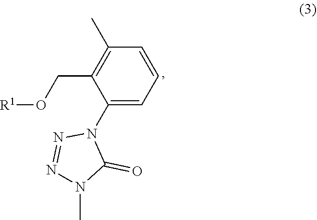

comprising the following steps:
a) reacting an azide compound with a compound represented by formula (1) to yield compound (2), compound (2) being a compound represented by formula (2) or its salts,

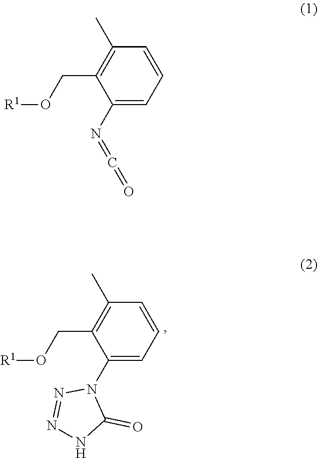

b) methylating compound (2) obtained in step a) using a methylating agent to obtain compound (3),

wherein R¹ denotes a hydrocarbon rest, and wherein in step a) a solvent system A and in step b) a solvent system B is used, wherein both solvent systems A and B comprise one dipolar aprotic solvent or a mixture of dipolar aprotic solvents as the main component.

The nature of alkyl rest R¹ is normally not critical for carrying out the reaction. For example, R¹ can be a C₁ to a C₁₀ rest or a C₁ to C₆ rest. In one embodiment, R¹ is selected from C₁-C₆ alkyl, C₃ to C₆ cycloalkyl and phenyl. Preferably, R¹ is C₁ to C₆ alkyl. More preferably, R¹ is selected from methyl or ethyl. Especially preferably, R¹ is methyl.

In one embodiment, R¹ is a hydrocarbon rest different from methyl. For example, R¹ can be a C₂ to a C₁₀ rest or a C₂ to C₆ rest. In one embodiment, R¹ is selected from C₂-C₆ alkyl, C₃ to C₆ cycloalkyl and phenyl. Preferably, R¹ is C₂ to C₆ alkyl. In one embodiment, R¹ is ethyl.

When reference is made herein to compound (2), this shall, unless specified otherwise, refer to the protonated and the deprotonated form of compound (2).

The deprotonated form of compound (2) is represented by formula (2b), with M⁺ being a cation, typically a metal cation such as of sodium or potassium. Preferably, M⁺ is Na⁺.

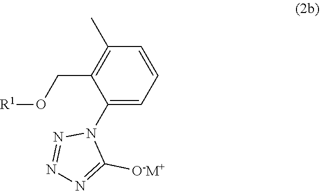

Suitable azide compounds are for example azide salts or organic azide compounds. Azide salts with metals different from ammonium, alkali or alkaline earth metals are normally less preferred. Preferred azide salts are azide salts of ammonium, alkali metals, such as lithium azide, sodium azide or potassium azide. The ammonium azide can notably be a quaternary ammonium azide salt, such as a tetraalkylammonium azide. Examples of tetraalkylammonium azides are tetramethylammonium azide, tetraethylammonium azide, and tetrabutylammonium azide. Preferred organic azides include silyl azides, such as trialkylsilyl azide, e.g. trimethylsilyl azide.

Preferably, said azide compound is selected from sodium azide, potassium azide and silyl azides. It is possible that such azide compounds do not always react directly with compound (1), but only indirectly, for example via intermediate derivatization or activation by a Lewis acid. In case an azide compound reacts only indirectly with compound (1), this shall still be considered to be a reaction of such azide compound with compound (1).

Typically, said azide compound is used in step a) in an amount of 0.9 to 3.0 molar equivalents relative to compound (1). Preferably said azide compound in an amount of 1.0 to 1.5 molar equivalents, more preferably 1.0 to 1.1 and even more preferably 1.03 to 1.08 molar equivalents, in each case relative to compound (1).

In one embodiment, step a) is carried out in the presence of a Lewis acid.

A Lewis acid is a chemical species that contains an empty orbital which is capable of accepting an electron pair from a Lewis base to form a Lewis adduct.

Lewis acids in the sense of this invention include for example aluminum chloride and silyl compounds represented by formula (4),

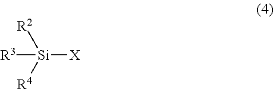

wherein R², R³ and R⁴ are independently a hydrocarbon rest and X is for example selected from Cl, Br or azide.

In one embodiment, said Lewis acid is selected from aluminum chloride and silyl compounds represented by formula (4),

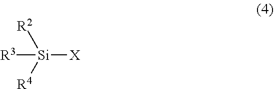

wherein R², R³ and R⁴ are independently a hydrocarbon rest and X is selected from Cl, Br or azide.

In one preferred embodiment, said Lewis acid is a silyl compound according to formula (4). Rests R², R³ and R⁴ can for example independently be aryl, alkyl, aralkyl rests. Preferably R², R³ and R⁴ are alkyl rests, more preferably C₁ to C₄ alkyl. Especially preferably R², R³ and R⁴ are methyl or ethyl. Particularly preferably R², R³ and R⁴ are methyl.

Preferred examples of silyl compounds (4) are trimethylsilyl chloride and trimethylsilyl azide.

In one preferred embodiment, step a) is carried out in the presence of an azide salt, e.g. an azide alkali salt, and in the presence of a catalytic amount of a silyl compound (4). It is assumed that azide salts and silyl halides form silyl azides, the latter being the species that undergo the cycloaddition reaction with the isocyanate group in compound (1).

Typically, silyl derivatives according to formula (4) are present in the reaction mixture in an amount of 0.005 to 1.0 molar equivalents relative to compound (1). Preferably, silyl derivatives according to formula (4) are present in the reaction mixture in an amount of 0.005 to 0.1 molar equivalents, more preferably 0.01 to 0.05 and even more preferably 0.01 to 0.025 molar equivalents relative to compound (1).

The use of silyl derivatives (4) allows for an efficient and safe process for making compound (2). In particular, such silyl derivatives are easier to handle and are less hazardous than other Lewis acids like aluminium chloride. Also the catalytic use avoids the use or large excess amounts of expensive reagents.

In a less preferred embodiment, said Lewis acid is aluminum chloride. Aluminum chloride turned out to be difficult to handle and to dose, since it is a solid and very hygroscopic. Also, the addition of aluminum chloride to dipolar protic solvents like DMF results in a strongly exothermic reaction leading to significant generation of heat. Furthermore, aluminum chloride produces significant amounts of solid byproducts that can cause problems during workup and are often difficult to filter.

In case aluminum chloride is used as Lewis acid, it is typically present in the reaction mixture in an amount of 0.01 to 1.0 molar equivalents relative to compound (1). Preferably, aluminum chloride is present in the reaction mixture in an amount of 0.01 to 0.5 molar equivalents, more preferably 0.05 to 0.25 and even more preferably 0.05 to 0.15 molar equivalents relative to compound (1).

According to the invention, solvent system A in step a) and solvent system B in step b) comprise a dipolar aprotic solvent as the main component. Solvent system A shall mean the solvent system present at the beginning of the dosage of compound (1) or, in case compound (1) is not being continuously added, at the beginning of the cycloaddition reaction. Solvent system B shall mean the solvent system present at the beginning of the dosage of the methylating agent in case the methylating agent is not being continuously added, at the beginning of the methylation reaction.

The term “main component” shall mean that said solvent system does not comprise any other solvent in a higher amount. In case two or more solvents are comprised in the same amount, they shall all be regarded as the main component. Compounds (1), (2) and (3) and any methylating agents are not considered solvents.

In the sense of this invention dipolar aprotic solvents are solvents with a relative permittivity (also referred to as “dielectric constant”) of 25 or above, and a sizable permanent dipole moment of 3 Debye or above, that cannot donate suitably labile hydrogen atoms to form strong hydrogen bonds.

In the sense of this invention, ketones like acetone, methyl isobutyl ketone or methyl ethyl ketone, esters like ethyl acetate and lactones like gamma-butyrolactone, which could undergo keto-enol-tautomerism, are considered protic solvents.

Examples of suitable dipolar aprotic solvents for both steps a) and b) are dimethylformamide (DMF), dimethylacetamide, N-methyl-2-pyrrolidone, N-ethyl-2-pyrrolidone, N-butyl-2-pyrrolidone, acetonitrile, hexamethyl phosphoramide, dimethylsulfoxide, dimethylpropylene urea, 1,3-dimethyl-2-imidazolidinone, 4-Methyl-1,3-dioxolan-2-one and combinations thereof.

Preferred dipolar aprotic solvents for both step a) and step b) are dimethylformamide, dimethylacetamide, N-methylpyrrolidone and combinations thereof. Dimethylformamide is especially preferred in step a) and b).

Typically, step a) is carried out such that solvent system A, the azide compound, compound (1) and optionally the Lewis acid are mixed in a reactor and brought to reaction at elevated temperature under stirring.

Solvent system A comprises one dipolar aprotic solvent or a mixture of dipolar aprotic solvents as the main component.

Preferably, a reactor is charged with solvent system A, the azide compound and optionally a Lewis acid. Typically, said mixture comprises 50 to 1000 g of dipolar aprotic solvent per mole of compound (1). To this mixture compound (1) is then added under stirring at elevated temperature. The dosage time of compound (1) is typically from 0.25 to 15 h, preferably 0.5 to 10 h, more preferably 1 to 5 h and even more preferably 1 to 3 h.

Compound (1) is normally added as a pure, undissolved compound. After complete addition of compound (1) it may be beneficial to stir the mixture at elevated temperature for some time (typically some hours).

Typical reaction temperatures for step a) are from 50 to 150° C., preferably 60 to 130° C., more preferably 70 to 120° C. and particularly 80 to 110° C.

When reference is made herein to compound (2), this shall, unless specified otherwise, refer to the protonated and the deprotonated form of compound (2).

After completion of the dosage of compound (1) to the reaction mixture, the mixture is typically let to react under the reaction conditions for an additional 0.5 to 15 h, preferably 1 to 7 h, more preferably 2 to 5 h and even more preferably 3 to 4 h. The post dosage reaction conditions may be identical to the reaction during dosage or may be varied within the ranges as defined above.

After completion of the cycloaddition reaction, it is advisable to destroy any residual azide groups. This can for example be done through addition of nitrite salts such as sodium nitrite and adjusting an acidic pH. For example, nitrite salts may be added in an amount of 0.01 to 1.5 mol, preferably 0,05 to 1.0 mol, more preferably 0.05 to 0.5 and more preferably 0.05 to 0.3 mol of nitrite per mol of azide originally employed.

Due to the adjustment of an acidic pH, the neutral undeprotonated form of compound (2) is formed.

To maintain the protonated form of compound (2) in solution, a solvent capable of dissolving protonated compound (2), such as Solvesso or toluene may be added before, during or after acidification of the reaction mixture, followed by a phase separation.

In one preferred embodiment, steps a) and b) are carried without isolating the compound (2) obtained in step a).

“Without isolating” shall mean in this context that compound (2) is not worked up to a solid product.

In one preferred embodiment, compound (2) is not worked up to a solid product that contains less than 50 wt % of water or solvents, based on the solid product.

For the subsequent methylation of compound (2), it is advantageous if compound (2) is at least partly present in its deprotonated form (2b).

In one embodiment, the organic phase is then extracted with an aqueous solvent, preferably water, having a basic pH, for example using an aqueous hydroxide solution, followed by phase separation. The so obtained aqueous phase contains compound (2b) and a part of the dipolar aprotic solvent of solvent system A.

According to the invention, the methylation reaction of compound (2) is carried out in solvent system B that comprises one dipolar aprotic solvent or a mixture of dipolar aprotic solvents as the main component. Especially preferred solvents for solvent system B are dimethylformamide, dimethylacetamide, N-methylpyrrolidone and combinations thereof. Especially preferred is dimethylformamide.

In case compound (2b) is contained in an aqueous phase resulting from aqueous extraction as described above, it may be necessary to add dipolar aprotic solvent and/or to remove water, e.g. by distillation methods.

The use of dipolar aprotic solvents in solvent system B has surprisingly turned out to be beneficial for improving the yield and the purity, especially the regioselectivity of the methylation reaction, thus leading to low amounts of methoxytetrazoles or of methylation products that have been methylated in other positions, e.g. the 2-position of the tetrazole ring.

Step b) is typically carried out such that compound (2b) is provided dissolved in solvent system B that comprises one dipolar aprotic solvent or a mixture of dipolar aprotic solvents as the main component. To this solution, a methylating agent is added. Preferably, said methylating agent is added over a duration of 0.1 to 15 h, preferably 1 to 6 h, more preferably 2 to 5 h.

Typically, in step b) 500 g to 2000 g of dipolar aprotic solvents are employed per mole of compound (2). Preferably, in step b) 700 g to 1500 g and more preferably 800 g to 1100 g of dipolar aprotic solvents are employed per mole of compound (2).

While solvent system B comprises one dipolar aprotic solvent or a mixture of dipolar aprotic solvents as the main component, it may comprise 0 to 40 wt % (based on solvent system B) of water. In one embodiment, solvent system B comprises 25 to 35 wt % (based on solvent system B) of water. In another embodiment, solvent system B comprises 0 to 10 wt % (based on solvent system B) of water.

In one embodiment, solvent system A and solvent system B comprise the same dipolar aprotic solvent or a mixture of the same dipolar aprotic solvents as the main component.

In one embodiment, solvent system A and solvent system B comprise the same dipolar aprotic solvent or the same mixture of dipolar aprotic solvents as the main component.

In one embodiment, the content by weight of each of the dipolar aprotic solvents comprised in solvents systems A and B differs by not more than 10 wt % relative to the solvent systems.

In one embodiment, solvent system A and solvent system B are identical with respect to the dipolar aprotic solvents contained in solvent system A and B.

By using similar or identical solvent systems A and B, the direct use of the reaction product of step a) in step b) without having to isolate compound (2) is facilitated.

Typically, the temperature of the reaction mixture is maintained from −20 to 70° C. throughout the methylation reaction, preferably from −5° C. to 25° C., more preferably from 10° C. to 22° C. The pH of the reaction mixture is preferably maintained from 3 to 14, preferably from 5 to 11, more preferably from 6.5 to 9 and even more preferably from 6.5 to 8.5. Typically, a base is continuously added to the reaction mixture to maintain the pH at a more constant level. The nature of the base used here is in in principle not critical. Suitable bases include organic bases (such as trimethylamine, pyridine, N-methyl morpholine, N-methyl piperidine, 4-dimethylaminopyridine, diisopropyl ethylamine, lutidine, collidine, diazabicycloundecene, diazabicyclononene), alkali metal carbonates (such as lithium carbonate, sodium carbonate, potassium carbonate, cesium carbonate), alkali metal hydrogen carbonates (such as lithium hydrogen carbonate, sodium hydrogen carbonate, potassium hydrogen carbonate, cesium hydrogen carbonate, alkali metal hydroxides (such as lithium hydroxide, sodium hydroxide, potassium hydroxide, cesium hydroxide), earth alkali metal hydroxides (such as magnesium hydroxide or calcium hydroxide), alkali metal hydrides (such as sodium hydride, potassium hydrides, or alkyl alkoxides (such as sodium tert.-butoxide, potassium ter.-butoxide. Potassium carbonate, cesium carbonate, sodium hydroxide and potassium hydroxide are preferred. Sodium hydroxide and potassium hydroxide are most preferred. Sodium hydroxide is particularly preferred. Quite surprisingly, it has turned out that the regioselectivity of the methylation can be increased when alkali metal hydroxides such as sodium hydroxide are used as a base.

In particular the use of alkali metal hydroxides as a base with DMF as the solvent or the main component of solvent system B has turned out to be beneficial in step b).

Preferred methylating agents are methyl halides (such as methyl chloride, methyl bromide or methyl iodide), methyl triflate, dimethyl sulfonate, methyl sulfuric acid esters or aryl sulfuric acid esters (such as methyl p-toluene sulfonate or methyl methane sulfonate). Preferably dimethyl sulphate is used as the methylating agent.

Typically, 0.9 to 3.0 molar equivalents of the methylating agent are employed in step b) relative to compound (2). Preferably 1.0 to 2.0 molar equivalents and more preferably 1.1 to 1.6 molar equivalents of the methylating agent are employed in step b) relative to compound (2).

After the completion of the dosage of methylating agent, the reaction mixture is in one embodiment let to react under the reaction conditions for typically up to 15 h, preferably up to 5 h, more preferably up to 2 h. The post dosage reaction conditions may be identical to the reaction during dosage of the methylating agent or may be varied within the ranges as defined above.

In a preferred embodiment, any residual methylating agent is decomposed after the completion of the methylation reaction. Typically, this is done through the addition of a suitable base. Typical bases suitable for destroying such methylation agents include organic amines, alkanolamines (e.g. ethanolamine), hydroxide salts (such as sodium hydroxide, potassium hydroxide, cesium hydroxide) and carbonate salts (such as sodium carbonate, potassium carbonate). Preferred bases are sodium hydroxide and potassium hydroxide. Preferably, such base is applied in liquid form, for example as an aqueous solution.

In a preferred embodiment, the same base is used during the aqueous extraction of compound (2b) as described above, if applicable, and the decomposition of excess methylating agent.

For the workup of compound (3), the product of the methylation reaction in step b) can for example be precipitated and filtered. The precipitation of compound (3) for example can be promoted by cooling of the reaction mixture and/or the addition of water or other solvents in which compound (3) is not or only poorly soluble.

Especially in case the precipitation of compound (3) has been supported by the addition of water, the filtered precipitate of compound (3) may contain significant amounts of water, water soluble impurities and/or other solvents from solvent system B.

In one embodiment, the filtrated precipitate of compound (3) is dried by removing remaining solvents including water in vacuo. The so obtained solid compound (3) can be essentially free from solvents and may be used for further synthesis. It is possible, however, that the product so obtained may contain water soluble impurities.

In one embodiment, the filtrated precipitate of compound (3) is treated with solvents capable of dissolving compound (3) and being immiscible with water, such as unsubstituted or substituted aliphatic or aromatic hydrocarbons or mixtures thereof, for example C6-C8 aliphatic hydrocarbons like hexane, heptane and octane and their isomers, cyclohexane, Solvesso, toluene, ethylbenzene, chlorobenzene or xylene. Thereby, a solution of compound (3) in said solvent, preferably aromatic hydrocarbons like toluene, ethylbenzene, xylene or chlorobenzene, is obtained. Most preferred solvent is xylene. Depending on the previous treatment of the product, an aqueous phase may be formed that contains any water and water soluble impurities. The aqueous phase and the solvent phase are then subjected to phase separation. Said phase separation can in one embodiment be carried out at elevated temperature depending on the boiling point of the solvent used. Compound (3) can so be obtained in a very high purity.

In case the precipitated and filtered compound (3) is treated with a solvent capable of dissolving compound (3) and immiscible with water, preferably aromatic hydrocarbons like toluene, ethylbenzene, xylene or chlorobenzene, it is possible to use the so obtained solution of compound (3) in further synthesis steps.

In one embodiment, the solution of compound (3) in said solvent, preferably xylene, is further concentrated by distillative removal of said solvent, preferably xylene. In one embodiment, the content of said solvent, preferably xylene, is reduced until a solid product with a solvent, preferably xylene, content of 20 to 60 wt % is obtained. Preferably the solvent, preferably xylene content is 30 to 55 wt %. The so obtained solid product containing compound (3) and solvent, preferably xylene, may then be used in further synthesis steps.

In one embodiment, processes according to the invention comprise the following steps
a) reacting an azide compound with compound (1) represented by formula (1) to yield compound (2) in a process comprising the following substeps:
   a1) providing a mixture of said azide compound and optionally a Lewis acid in solvent system A,
   a2) adding compound (1),
   a3) optionally destroying excess azide compound,
b) methylating compound (2) obtained in step a) using a methylating agent to obtain compound (3), comprising the following substeps:
   b1) providing a solution of compound (2) obtained in step a) in a solvent system B,
   b2) adding a methylating agent,
   b3) optionally destroying excess methylating agent,
   b4) working up compound (3).

In one embodiment, processes according to the invention comprise the following steps
a) reacting an azide compound with compound (1) represented by formula (1) to yield compound (2) in a process comprising the following substeps:
   a1) providing a mixture of said azide compound and optionally a Lewis acid in solvent system A,
   a2) adding compound (1),
   a3) optionally destroying excess azide compound,
   a4) optionally extracting compound (2) obtained in the previous substeps using an aqueous solvent having a basic pH,
   a5) optionally adding further dipolar aprotic solvent and/or removing water from the mixture obtained in step a4), for example by distillative methods, such that dipolar, aprotic solvents are the main component of said solvent mixture to be used in the following methylation reaction (solvent system B),
b) methylating compound (2) obtained in step a) using a methylating agent to obtain compound (3), comprising the following substeps:
   b1) providing a solution of compound 2 obtained in step a) in a solvent system B,
   b2) adding a methylating agent,
   b3) optionally destroying excess methylating agent,
   b4) working up compound (3).

In one embodiment, processes according to the invention comprise the following steps
a) reacting an azide compound with compound (1) represented by formula (1) to yield compound (2) in a process comprising the following substeps:
   a1) providing a mixture of said azide compound and optionally a Lewis acid in solvent system A,
   a2) adding compound (1),
   a3) optionally destroying excess azide compound,
   a4) optionally extracting compound (2) obtained in the previous substeps using an aqueous solvent having a basic pH,
   a5) optionally adding further dipolar aprotic solvent and/or removing water from the mixture obtained in step a4), for example by distillative methods, such that dipolar, aprotic solvents are the main component of said solvent mixture to be used in the following methylation reaction (solvent system B),
b) methylating compound (2) obtained in step a) using a methylating agent to obtain compound (3), comprising the following substeps:
   b1) providing a solution of compound 2 obtained in step a) in a solvent system B,
   b2) adding a methylating agent,
   b3) optionally destroying excess methylating agent,
   b4) working up compound (3) by precipitating compound (3), followed by filtration,
   B5) optionally treating the precipitate from step b4) with unpolar solvent, preferably xylene, separating any aqueous phase present and removing s a portion or all of said unpolar solvent.

In one embodiment, processes according to the invention comprise the following steps
a) reacting an azide compound with compound (1) represented by formula (1) to yield compound (2) in a process comprising the following substeps:
   a1) providing a mixture of said azide compound and optionally a Lewis acid in solvent system A,
   a2) adding compound (1),
   a3) destroying excess azide compound,
   a4) extracting compound (2) obtained in the previous substeps using an aqueous solvent having a basic pH,
   a5) adding further dipolar aprotic solvent and/or removing water from the mixture obtained in step a4), for example by distillative methods, such that dipolar, aprotic solvents are the main component of said solvent mixture to be used in the following methylation reaction (solvent system B),
b) methylating compound (2) obtained in step a) using a methylating agent to obtain compound (3), comprising the following substeps:
   b1) providing a solution of compound 2 obtained in step a) in a solvent system B,
   b2) adding a methylating agent,
   b3) destroying excess methylating agent,
   b4) working up compound (3) by precipitating compound (3), followed by filtration,
   B5) optionally treating the precipitate from step b4) with unpolar solvent, preferably xylene, separating any aqueous phase present and removing s a portion or all of said unpolar solvent.

In one embodiment, processes according to the invention comprise the following steps
a) reacting an azide compound with compound (1) represented by formula (1) to yield compound (2) in a process comprising the following substeps:
   a1) providing a mixture of said azide compound and optionally a Lewis acid in solvent system A,
   a2) adding compound (1),
   a3) destroying excess azide compound,
   a4) extracting compound (2) obtained in the previous substeps using an aqueous solvent having a basic pH
   a5) adding further dipolar aprotic solvent and/or removing water from the mixture obtained in step a4), for example by distillative methods, such that dipolar, aprotic solvents are the main component of said solvent mixture to be used in the following methylation reaction (solvent system B),
b) methylating compound (2) obtained in step a) using a methylating agent to obtain compound (3), comprising the following substeps:
   b1) providing a solution of compound 2 obtained in step a) in a solvent system B,
   b2) adding a methylating agent,
   b3) destroying excess methylating agent,
   b4) working up compound (3) by precipitating compound (3), followed by filtration,
   B5) treating the precipitate from step b4) with unpolar solvent, preferably xylene, separating any aqueous phase present and removing s a portion or all of said unpolar solvent.

Processes according to the invention are easy and economical to carry out. They require only a small number of reagents and minimal requirements with respect to equipment and are environmentally friendly.

Processes according to the invention can be carried out without isolating compound (2) after step a), thus allowing a very efficient synthesis of compound (3).

Processes according to the invention allow the manufacture of compound (3) in high yields and in high purity. In particular, a high content of the isomer represented by formula (3) can be obtained with low contents of undesired other position isomers, such as compounds (5a) and (5b).

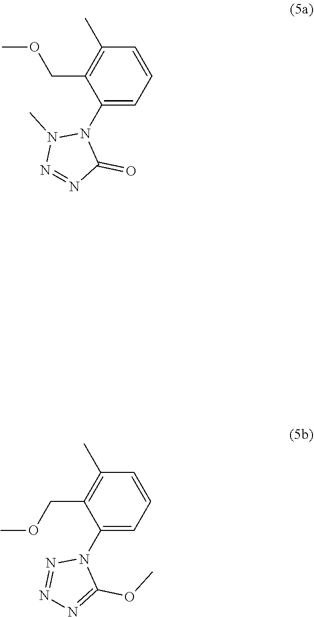

### EXAMPLE

### Example 1: Preparation of Compound (3) Via Non-Isolated Intermediate Compound (2)

In a stirred reactor 119.4 g (1.82 mol) of sodium azide (99.0%) was precharged at 25° C. followed by 317.1 g of DMF. Then 4.7 g (0.043 mol) of chlorotrimethylsilane (99.0%) was added. The suspension was heated up to 90° C. and stirred 30 min at 90° C. Subsequently, 310.0 g (1.73 mol) of 1-isocyanato-2-(methoxymethyl)-3-methyl-benzene (Compound (1)) (99.0%) was dosed over 2 h at 90° C. Then the suspension was stirred further 4 h at 90° C. In a second reactor 29.9 g (0.17 mol) of a 40% aqueous NaNO₂ solution, 109.7 g water and 417.9 g of toluene are precharged at 25° C. and the reactor content from the first reactor is transferred. For decomposition of residual sodium azide 350.8 g of HCl (20%) was dosed evenly over 70 min keeping the temperature at 45° C. After that 835.8 g of additional toluene was added. Then 103.0 g (0.13 mol) of a 12% aqueous solution of sulfamic acid was added at 45° C. The phases were separated at 45° C. Subsequently, the organic phase was extracted with 706.3 g (1.77 mol) of a 10% aqueous NaOH solution at 45° C. During the extraction the pH is kept at 11. Then the phases were separated at 45° C. After pH adjustment of the water phase to 8 by adding of 3.6 g of HCl (20%), 1271.9 g of water phase with a content of 32.1% sodium;1-[2-(methoxymethyl)-3-methyl-phenyl]tetrazol-5-olate (Compound (2b)) was obtained.

1263 g (1.68 mol) of this water phase was transferred to a stirred reactor equipped with a fractionating column. 1319 g of DMF was added and 585 g of a DMF/water mixture was distilled off (inner temperature up to 53° C./35 mbar). After cooling down the remaining reaction mixture to 17° C., 299 g (2.35 mol) dimethyl sulphate (99%) was dosed in 3 h. The pH of the reaction mixture was being kept between 7-8 with NaOH (25% in water). After the dosing was completed, the mixture was post-reacted for 30 min at 18° C. Then the mixture was transferred into another stirred reactor. 134 g of sodium hydroxide (25% in water) was added and the mixture was heated to 50° C. for 3 h to decompose excess of dimethyl sulphate. For precipitation of the product the mixture was heated to 55° C. and 2952 g of water was added. Subsequently the suspension was cooled down to 0° C. The product was separated by pressure filtration and the wet filter cake was washed twice with water. After drying of the filter cake at 80° C. under reduced pressure (20 mbar), 360 g (1.50 mol) of compound 3 with a purity of 97.8% (w/w HPLC) was isolated as off-white solid with an overall yield of 86.9% relative to compound (1). The ratio of compounds (3) : (5a) : (5b) was 99.9:0:0.1.

### Example 2: Manufacture of Compound (3) Via Non-Isolated Compound (2)

In a stirred reactor 118.0 g (1.80 mol) of sodium azide (99.0%) was precharged at 25° C. followed by 312.8 g of DMF. Then 4.7 g (0.043 mol) of chlorotrimethylsilane (99.0%) was added. The suspension was heated up to 90° C. and stirred 30 min at 90° C. Subsequently, 330.0 g (1.71 mol) of 1-isocyanato-2-(methoxymethyl)-3-methyl-benzene (Compound (1)) (91.9%) was dosed over 2 h at 90° C. Then the suspension was stirred further 4 h at 90° C. In a second reactor 29.5 g (0.17 mol) of a 40% aqueous NaNO₂ solution, 108.4 g water and 413 g of toluene are precharged at 25° C. and the reactor content from the first reactor is transferred. For decomposition of residual sodium azide 331.1 g of HCl (20%) was dosed evenly over 70 min keeping the temperature at 45° C. After that 826 g of additional toluene was added. Then 101.8 g (0.13 mol) of a 12% aqueous solution of sulfamic acid was added at 45° C. The phases were separated at 45° C. Subsequently, the organic phase was extracted with 692.3 g of a 10% aqueous NaOH solution at 45° C. During the extraction the pH is kept at 11. Then the phases were separated at 45° C. After pH adjustment of the water phase to 8 by adding of 4.8 g of HCl (20%), 1268 g of water phase with a content of 30.2% Compound (2b) was obtained.

The water phase was mixed with 1372 g DMF and 79 g water and cooled to 10° C., before 315 g dimethyl sulfate were added within 3 h. The pH of the reaction mixture was being kept between 7-8 with NaOH (25% in water). After the dosing was completed, the mixture was post-reacted for 30 min at 15° C. Then the mixture was transferred into another stirred reactor. 182 g of sodium hydroxide (25% in water) was added and the mixture was heated to 50° C. for 2 h to decompose excess of dimethyl sulphate. For precipitation of the product the mixture was heated to 55° C. and 1635 g of water was added. Subsequently the suspension was cooled down to 0° C. The product was separated by pressure filtration and the wet filter cake was washed twice with water. After drying of the filter cake at 80° C. under reduced pressure (20 mbar), 368 g (1.37 mol) of compound 3 with a purity of 87.3% (w/w HPLC) was isolated as off-white solid with an overall yield of 80.2% relative to compound (1). No undesired isomers (5a) or (5b) were obtained.

### Example 3: Preparation of Compound (3) Via Non-Isolated Intermediate Compound (2)

In a stirred reactor 118.0 g (1.80 mol) of sodium azide (99.0%) was precharged at 25° C. followed by 312.8 g of DMF. Then 4.7 g (0.043 mol) of chlorotrimethylsilane (99.0%) was added and the agitator was started. The suspension was heated up to 90° C. and stirred 30 min at 90° C. Subsequently, 330.0 g (1.71 mol) of 1-isocyanato-2-(methoxymethyl)-3-methyl-benzene (91.9%) was dosed over 3 h at 90° C. Then the suspension was stirred further 2 h at 90° C. In a second reactor 29.5 g (0.17 mol) of a 40% aqueous NaNO₂ solution, 108.4 g water and 413.1 g of toluene are precharged at 25° C. and the reactor content from the first reactor is transferred. For decomposition of residual sodium azide 326.4 g of HCl (20%) was dosed evenly over 70 min keeping the temperature at 45° C. After that 826.1 g of additional toluene was added. Then 101.8 g (0.126 mol) of a 12% aqueous solution of sulfamic acid was added at 45° C. The phases were separated at 45° C. Subsequently, the organic phase was extracted with 720.2 g of a 10% aqueous NaOH solution at 45° C. During the extraction the pH is kept at 11. Then the phases were separated at 45° C. After pH adjustment of the water phase to 8 by adding of 4.5 g of HCl (20%), 1239 g of water phase with a content of 31.2% sodium;1-[2-(methoxymethyl)-3-methylphenyl]tetrazol-5-olate 2b was obtained.

1229 g (1.58 mol) of this water phase was transferred to a stirred reactor equipped with a fractionating column. 1249 g of DMF was added and 580 g of a DMF/water mixture was distilled off (inner temperature up to 53° C./35 mbar). After cooling down the remaining reaction mixture to 17° C., 282 g (2.22 mol) dimethyl sulphate (99%) was dosed in 3 h. The pH of the reaction mixture was being kept between 7-8 with NaOH (25% in water). After the dosing was completed, the mixture was post-reacted for 30 min at 18° C. Then the mixture was transferred into another stirred reactor 127 g of sodium hydroxide (25% in water) was added and the mixture was heated to 50° C. for 3 h to decompose excess of dimethyl sulphate. For precipitation of the product the mixture was heated to 55° C. and 2790 g of water was added. Subsequently the suspension was cooled down to 0° C. The product was separated by pressure filtration and the wet filter cake was washed twice with water. 695 g of a wet filter cake was obtained.

For comparison reasons, 100 g of this wet filter cake was dried at 80° C. under reduced pressure (20 mbar) to give 50.4 g (0.21 mol) of compound 3 with a purity of 93.2% (w/w H PLC) as offwhite solid. Extrapolated to the whole filter cake, this correlates to an overall yield of 86.2% relative to compound (1). No undesired isomers (5a) or (5b) were obtained.

Additionally, 583.6 g of the wet filter cake was transferred into a reactor and diluted with 652 g xylene. Under agitation the suspension was heated to 58° C. and the phases were separated. The organic phase was partly concentrated at 85° C. (100 mbar) to yield 565.3 g (1.20 mol) of a melt comprising xylene and compound (3) as a light yellow solid (content of compound (3) of 49.5%). Extrapolated to the whole filter cake, this correlates to an overall yield of 83.3% relative to compound (1). No undesired isomers (5a) or (5b) were obtained.

### Example 4: Preparation of Compound (3) in Acetone and Potassium Carbonate as Base Via Isolated Intermediate Compound (2)

In a stirred reactor 15.0 g (67 mmol) of 4-[2-(methoxymethyl)-3-methyl-phenyl]-1H-tetrazol-5-one (compound (2)) was precharged at 25° C. followed by 59 g of acetone and 18.5 g (134 mmol) of potassium carbonate. After cooling down the remaining reaction mixture to 10° C., 13.4 g (100 mmol) dimethyl sulphate (99%) was dosed in 2 h. After the dosing was completed, the mixture was allowed to warm to 23° C. and was post-reacted for 20 h at 23° C.

After 30 g of xylene was added to the reaction mixture, the acetone was distilled off, and the sump was washed with a sodium hydroxide aqueous solution and water at 70° C. The water was removed from the organic layer by reflux dehydration. To the remaining sump 30 g of n-heptane was added at 75° C. and the solution was gradually cooled down under stirring to 0° C. (temperature ramp of 10 K/h), followed by post-stirring time of 14 h at 0° C. The resulting slurry was filtered, and the crystal was washed twice with 15 mL of a 1:1 w % solution of xylene and n- heptane. After drying under reduced vacuum (20 mbar, 80° C., 4 h) 12.9 g (78.8% yield) of compound (3) was isolated.

### Example 5: Preparation of Compound (3) in DMF and Potassium Carbonate as Base Via Isolated Intermediate Compound (2)

In a stirred reactor 15.0 g (67 mmol) of 4-[2-(methoxymethyl)-3-methyl-phenyl]-1H-tetrazol-5-one (compound (2)) was precharged at 25° C. followed by 59 g of DMF and 18.5 g (134 mmol) of potassium carbonate. After cooling down the remaining reaction mixture to 10° C., 13.4 g (100 mmol) dimethyl sulphate (99%) was dosed in 2 h. After the dosing was completed, the mixture was allowed to warm to 23° C. and was post-reacted for 20 h at 23° C. 16 g of 25% aqueous sodium hydroxide solution was added, and the reaction mixture was heated to 55° c for 2 h. At this temperature 146 g of water was added, and the solution was gradually cooled down under stirring to 0° C. (temperature ramp of 10 K/h), followed by post-stirring time of 14 h at 0° C. The resulting slurry was filtered, and the crystal was washed twice with 37 mL of water. After drying under reduced vacuum (20 mbar, 80° C., 4 h) 13.2 g (80.1% yield) of compound (3) was isolated.

### Example 6: Preparation of Compound (3) in DMF and Sodium Hydroxide as Base Via Isolated Intermediate Compound (2)

In a stirred reactor 15.0 g (67 mmol) of 4-[2-(methoxymethyl)-3-methyl-phenyl]-1H-tetrazol-5-one (compound (2)) was precharged at 25° C. followed by 59 g of DMF and 4 mL of water. After cooling down the remaining reaction mixture to 10° C., the pH was adjusted to pH 7.5 with 25% aq. sodium hydroxide solution. 13.4 g (100 mmol) dimethyl sulphate (99%) was dosed in 2 h to this 10° C. cold reaction mixture, while keeping the pH constant at pH 7.5 by addition of 25% aqueous sodium hydroxide solution. After the dosing was completed, the mixture was allowed to warm to 23° C. and was post-reacted for 20 h at 23° C. 16 g of 25% aq. sodium hydroxide solution was added, and the reaction mixture was heated to 55° C. for 2 h. At this temperature 146 g of water was added, and the solution was gradually cooled down under stirring to 0° C. (temperature ramp of 10 K/h), followed by post-stirring time of 14 h at 0° C. The resulting slurry was filtered, and the crystal was washed twice with 37 mL of water. After drying under reduced vacuum (20 mbar, 80° C., 4 h) 14.3 g (84.8% yield) of compound 3 was isolated.

The content of undesired isomers (5a) and (5b) in exampled 4 to 6 were determined by HPLC over a silica column.

### Overview of Results of Examples 4-6

| | | | | yield of | yield of |
|---|---|---|---|---|---|
| | | | isolated | undesired | undesired |
| | | | yield of | isomer | isomer |
| example no | solvent | base | compound 3 | (5a) | (5b) |
| 4 | acetone | K₂CO₃ | 78.8% | 0.37% | 0.09% |
| 5 | DMF | K₂CO₃ | 80.1% | 0% | 0% |
| 6 | DMF | NaOH | 84.8% | 0% | 0% |

1. A process for making a compound represented by a formula (3) 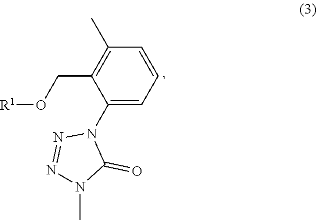
comprising:
(a) reacting an azide compound with a compound represented by formula (1) to yield compound (2), compound (2) being a compound represented by formula (2) or its salts, 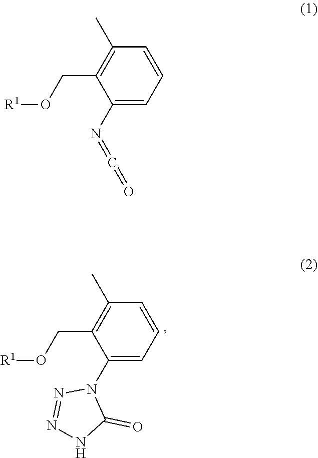
(b) methylating compound (2) obtained in step a) using a methylating agent to obtain compound (3),
wherein R¹ denotes a hydrocarbon rest, and wherein in step a) a solvent system A and in step b) a solvent system B is used, wherein both solvent systems A and B comprise one dipolar aprotic solvent or a mixture of dipolar aprotic solvents as the main component.

2. The process according to claim 1, wherein R¹ is methyl or ethyl.

3. The process according to claim 1, wherein solvent system A and solvent system B comprise the same dipolar aprotic solvent or a mixture of the same dipolar aprotic solvents as the main component.

4. The process according to claim 1, wherein said dipolar aprotic solvents are selected from dimethylformamide, dimethylacetamide, N-methylpyrrolidone, and combinations thereof.

5. The process according to claim 1, wherein steps a) and b) are carried without isolating the compound (2) obtained in step a).

6. The process according to claim 1, wherein step a) is carried out in the presence of a Lewis acid.

7. The process according to claim 6, wherein said Lewis acid is an alkali azide or a silyl compound represented by formula (4), 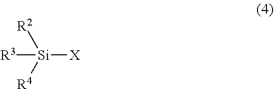
wherein R², R³ and R⁴ are independently a hydrocarbon rest and X is selected from Cl, Br, or azide.

8. The process according to claim 7 wherein said Lewis acid is a trialkylsilyl halide.

9. The process according to claim 6, wherein said Lewis acid, is used in a catalytic amount.

10. The process according to claim 7, wherein the alkali azide compound is sodium azide.

11. The process according to claim 1 wherein said methylating agent is selected from methyl halides, methyl triflate, dimethyl sulfonate, methyl sulfuric acid esters, or aryl sulfuric acid esters.

12. The process according to claim 8 wherein the trialkylsilyl halide is trimethylchlorosilane.

13. The process according to claim 7 wherein the compound (4) is trimethylsilyl azide.

14. The process according to claim 11 wherein the methylating agent is selected from methyl chloride, methyl bromide, methyl iodide, methyl p-toluene sulfonate, methyl methane sulfonate, or dimethyl sulphate.
